(19) United States
(12) Patent Application Publication
(10) Pub. No.: US 2012/0208255 A1
(43) Pub. Date: Aug. 16, 2012

(54) APPARATUS AND PROCESS FOR PRODUCTION OF AN ENCAPSULATED CELL PRODUCT

(75) Inventors: Lisa Beckler Andersen, Lakewood, CO (US); John H. Evans, IV, Superior, CO (US); Christine A. Singer, Lakewood, CO (US)

(73) Assignee: GEOSYNFUELS, LLC, Golden, CO (US)

(21) Appl. No.: 13/027,267

(22) Filed: Feb. 14, 2011

(51) Int. Cl. C12N 11/10; C12M 1/12; C12N 11/02

(52) U.S. Cl. 435/178; 435/177; 435/297.1

(57) ABSTRACT

A process for production of an encapsulated cell product, the process comprises the steps of concentrating cells from a propagation medium using a tangential flow filtration system. Mixing the concentrated cells with an encapsulation medium to form a cell encapsulation mixture. Polymerizing, gelling, or cross-linking the cell encapsulation mixture to form an encapsulated cell product.

### FIELD

The present patent document relates to an apparatus and process for encapsulation of cells in an encapsulation medium such as a polymer matrix.

### BACKGROUND

Although it is known that cells may be encapsulated in a polymer matrix, very little is known about trying to produce encapsulate cells on an industrial level. The scaling of any process often presents numerous obstacles and scaling the production of encapsulated cells is no different. While beakers and centrifuges may be sufficient to create a small quantity of entrapped cells in a laboratory, laboratory techniques and equipment can not be scaled to effectively produce large quantities of encapsulated cells for use in large scale industrial bioreactors.

Despite the advantages achieved by the use of encapsulated cells, there are no apparatuses or processes designed to produce an encapsulated cell product at large scale. For example, it would be beneficial to be able to produce encapsulated cell products to supply reactors at the 20,000 L (or 75,000 gal.) scale or a series of such reactors.

Nagashima et al. in Continuous Ethanol Fermentation Using Immobilized Yeast Cells published in 1984 in Biotechnology and Bioengineering, Volume 26, pages 992-997 describes production of yeast encapsulated in calcium alginate, but only to supply a relatively small (1000 L) research reactor. In their description, preparation of yeast encapsulated in calcium alginate beads “was carried out by showering drops of sodium alginate solution containing live yeast cells from the top nozzle into calcium chloride solution in the reactors. The preparation of cell beads was completed within several hours.” The apparatus and methods described Nagashima et al. would not be suitable for producing larger quantities of beads.

Similarly, commercially available systems for bead production of any size are limited. LentiKat's Biotechnologies advertises a commercial system for bead production, however, the LentiKat's system only provides for a small scale production suitable for small experiments and not for industrial production.

Furthermore, many of the processes used to encapsulate cells in a polymer matrix at the laboratory scale would not be practicable at large scale given their consumption of the products used to encapsulate the cells. Inefficient use of encapsulation medium and other supplies is not a big concern when done on a small scale. However, such inefficient use of resources may be extremely costly when scaled up. To this end, processes and apparatuses that can efficiently produce encapsulated cell products are needed. In addition, process and apparatuses that can be scaled up and effectively produce encapsulated cell products are needed.

### SUMMARY OF THE INVENTION

In view of the foregoing, an object according to one aspect of the present patent document is to provide improved apparatuses and processes for producing an encapsulated cell product. The apparatuses and processes may be used to produce encapsulated cell products on a large or small scale. Preferably the apparatuses and processes address, or at least ameliorate one or more of the problems described above. To this end, a process for production of an encapsulated cell product is provided. The process comprises the steps of: concentrating cells from a propagation medium using a tangential flow filtration system; mixing concentrated cells with an encapsulation medium to form a cell encapsulation mixture; and polymerizing the cell encapsulation mixture to form an encapsulated cell product.

In one embodiment of the processes described herein, the cells are concentrated in the retentate of the tangential flow filtration system. In embodiments that concentrate the cells in the retentate of the tangential flow filtration system, a portion of the retentate may be used as an inoculum for a subsequent production of an encapsulated cell product. In embodiments that include the use of retentate, the retentate may be cycled between a retentate holding vessel and the tangential flow filtration system. In certain embodiments, the retentate may be continuously cycled until the retentate achieves a cell concentration of greater than 180 grams wet mass of cells per liter.

In other embodiments, the mixing step is performed with a device that helps preserve the viability of the cells. In one embodiment a reciprocating shaker is used. In another embodiment a reciprocating disk is used.

In another aspect, a system for production of an encapsulated cell product is provided. The system comprises: a bioreactor; a tangential flow filtration system in communication with the bioreactor; and a retentate holding vessel in communication with the tangential flow filtration system.

In another embodiment of the system, the system further includes a reciprocating shaker operatively arranged to mix a cell suspension from the retentate holding vessel with an encapsulation medium.

In yet another embodiment of the system, the bioreactor is a chemostat bioreactor. A chemostat bioreactor is one type of bioreactor that allows embodiments of the system to be continuously operated.

In another embodiment, a process for production of an encapsulated cell product is provided. The process comprises the steps of: concentrating cells from a propagation medium; mixing the concentrated cells with an encapsulation medium using a reciprocating shaker to form a cell encapsulation mixture; and polymerizing the cell encapsulation mixture to form an encapsulated cell product.

In one embodiment of the process, the cells are concentrated to greater than 180 grams wet mass of cells per liter. In another embodiment, the encapsulation medium has a viscosity of about 1000 to 3500 centistokes (cSt.), or more preferably 2000 to 3000 centistokes (cSt.). In yet another embodiment, the cell encapsulation mixture has a viscosity of about 1000 to 3500 cSt. or more preferably 1500 to 2500 cSt.

In another aspect, a batch process for production of an encapsulated cell product is provided. The process comprising the steps of: propagating cells in a bioreactor to form a first batch of cells; concentrating cells from the first batch of cells; mixing concentrated cells with a encapsulation medium to form a cell encapsulation mixture; polymerizing the cell encapsulation mixture to form an encapsulated cell product; and propagating cells in a bioreactor to form a second batch of cells using cells from the first batch of cells as an inoculum.

In yet another embodiment, the bioreactor is not sterilized between propagating the first batch of cells and propagating the second batch of cells.

In another embodiment, the concentrating step is performed by a tangential flow filtration system and the cells are concentrated in a retentate. In embodiments including a tangential flow filtration system, a portion of the retentate may be used as the inoculum. In other embodiments, the retentate may be cycled between a retentate holding vessel and the tangential flow filtration system. In certain embodiments, the cycling step is performed until the retentate achieves a cell concentration of greater than 180 grams wet mass of cells per liter.

In yet another embodiment, the inoculum comprises about 5% to 10% of the volume of the first batch of cells.

In another aspect, a process for production of an encapsulated cell product is provided. The process comprises the steps of: mixing an aqueous solution with a concentrated encapsulation medium to form an encapsulation medium, wherein the mixing is performed without the addition of heat sufficient to sterilize the encapsulation medium; mixing cells with the encapsulation medium without heating to form a cell encapsulation mixture; and polymerizing the cell encapsulation mixture to produce an encapsulated cell product.

In one embodiment, a sterilization agent is added to the aqueous solution. The concentrated encapsulation medium is then sterilized via the aqueous solution when the two are mixed together. In certain of those embodiments, the sterilization agent is sodium chlorite.

In other embodiments, the process may be varied by the inclusion of various additional additives. For example, some embodiments include the additional step of adding antibiotics to the encapsulation medium. In other embodiments, nutrients are added to the encapsulation medium. In yet other embodiments, vitamins are added to the encapsulation medium.

In yet another embodiment, an additional step of irradiating the concentrated encapsulation medium is performed. Instead of or in addition to irradiating the concentrated encapsulation medium, the encapsulation medium may be irradiated.

In another embodiment, the concentrated encapsulation medium comprises sodium alginate. In one embodiment where the concentrated encapsulation medium comprises sodium alginate, the sodium alginate is mixed with the aqueous solution to produce an encapsulation medium with a viscosity of about 1000 to 3500 centistokes or more preferably 2000-3000 centistokes. In yet another embodiment the encapsulation medium has a sodium alginate concentration of 3.0% or less weight per volume, preferably 2.5% or less, and even more preferably about 2.0% weight per volume.

In another embodiment, the cell encapsulation mixture has a viscosity of about 1000 to 3500 centistokes or more preferably 1500 to 2500 centistokes.

In yet another aspect, an alginate bead for encapsulation of a cell is provided. The bead comprises: polymerized alginate in a concentration of 3% or less weight per volume; and an encapsulated cell. In other embodiments, the alginate concentration is preferably 2.5% or less weight per volume, and more preferably about 2.0% weight per volume.

The apparatuses and methods for producing entrapped cells described herein provide scalable solutions that may be used for mass production. Further aspects, objects, desirable features, and advantages of the devices and methods disclosed herein will be better understood from the detailed description and drawings that follow in which various embodiments are illustrated by way of example. It is to be expressly understood, however, that the drawings are for the purpose of illustration only and are not intended as a definition of the limits of the claimed embodiments.

### BRIEF DESCRIPTION OF THE DRAWINGS

FIG. 1 illustrates a general process for the production of an encapsulated cell product.

FIG. 2 illustrates a process for production of encapsulation medium from a dry encapsulation polymer.

FIG. 3A illustrates an embodiment of a process for producing a concentrated cell suspension.

FIG. 3B illustrates an embodiment of a system for production of a concentrated cell suspension as shown in FIG. 3A.

FIG. 4 is a partial cutaway view of an exemplary bioreactor for propagating cells.

### DETAILED DESCRIPTION OF THE PREFERRED EMBODIMENTS

Consistent with its ordinary biological meaning, the term “cell” is used herein to refer to the smallest unit of life that is a living thing. “Cell” includes both Prokaryotic and Eukaryotic cells. By way of example, “cell” includes but is not limited to bacteria, yeasts, fungal, algal, insect, or mammalian cells to name a few.

Cells that may be encapsulated include single cells, including all cells derived from any member of the plant, animal, fungal, protist, eubacteria or archaebacteria kingdoms of life. Single cells may be cells derived or removed from a living plant, animal, or fungus, otherwise known as primary cells. Primary cells can be derived, for example, from mammalian tissues, insect tissues, nematode tissues, Arabidopsis tissues, tomato plant tissues, or tobacco plant tissues. Primary cells can also be isolated from fungi or protists.

Single cells for encapsulation can also be derived from established cell culture lines. For example, cells can be derived from cultured cell lines such as the HeLa mammalian cell line, plant cell lines, algal cell lines, insect cell lines such as Sf9 cells, and cell lines from nematodes, insects, amphibians, reptiles, and other animals and plants.

Cells may also be manufactured. For example, cells may be derived from fusions of two different cells, such as hybridoma cells.

In addition to single cells, functional clusters of cells or cell tissues may be immobilized by encapsulation. Cell tissues could include any tissues derived from plants, protists, fungi or animals. For example, clusters of cells such as an acinus (a tissue) from a human pancreas or gap junctionally-connected or otherwise functionally-connected groups of neuronal and glial cells may be encapsulated.

The term “microbe” is used herein to refer to its ordinary meaning of an organism that is unicellular. As non-limiting examples, “microbe” includes yeasts, bacteria, fungi, archaea, protists, plankton and planarian to name a few. The term “microbe” is completely encompassed by the term “cell.”

The term “encapsulate” or “encapsulation” is used herein to refer to enclosing cells in an encapsulation medium. “Encapsulation” of cells is performed in an encapsulation medium that is porous enough to allow nutrients and other products needed by the cell to flow in, and byproducts produced by the cell to flow out, while preventing the cell from escaping. “Encapsulation” as used herein includes processes known generally as immobilization of cells, although one may immobilize cells in ways other than encapsulation, for example by adsorption to a substrate. More generally “encapsulation” includes any type of process that forms a casing or capsule around the cells including cell immobilization techniques.

Encapsulation media for encapsulation of cells may include both natural and synthetic materials. By way of non-limiting example, encapsulation media include numerous natural and synthetic polymers. Natural materials include alginate, a natural product from brown algae (seaweed), carrageenan, xanthan gums, chitosan, agarose, agar, collagen, cellulose and its derivatives, hyaluronate, pectin, fibrin, protein, nucleic acids and gelatin. Synthetic materials used for encapsulation include epoxy resin, photo cross-linkable resins, poly(vinyl alcohol), polyacrylamide, polyester, polystyrene, poly(acrylic acid), poly(ethylene oxide), polyphosphazene and polyurethane.

In some applications, two or more materials may be used as the encapsulation medium, for example, alginate and polyvinyl alcohol or copolymers of poly(ethylene oxide) and polypropylene oxide) or of poly(ethylene oxide) and poly(lactic acid) could be used as an encapsulating matrix.

Cells encapsulated in a polymer matrix may be used for many different industrial processes, including for example fermentation, and waste water treatment. Matrix encapsulation of cells includes numerous benefits over the use of ‘free’ cells. ‘Free’ cells are cells that are not encapsulated or immobilized in any way. One of the biggest advantages of encapsulated cell systems over ‘free’ cell systems is the high cell densities an encapsulated cell system can achieve. High cell densities are desirable in numerous different fermentative processes like the production of bio-based chemicals, such as 3-hydroxypropionic acid, glucaric acid, levulinic acid, xylitol, acetic acid, citric acid, lactic acid, ethanol, and the like. The high cell density achieved by matrix-encapsulated cell systems are beneficial for achieving high volumetric productivities, yet free cell systems are not able to achieve such high cell densities.

Another advantage of matrix-encapsulation of cells is that encapsulation facilitates continuous, rather than a batch, operation of biochemical processes, such as fermentation processes, without washout of the cellular biocatalysts. Continuous fermentation processes experience less down time compared to batch processes. Less down time lends an economic advantage to continuous systems. In addition to less down time, prevention of cell washout in a continuous system represents a great cost savings, as propagation of cells for an industrial process is expensive.

In certain applications, use of encapsulated cell systems provides advantages in addition to high cell density and prevention of washout. In applications where the medium contacting the cells contains compounds that are deleterious to the cells, encapsulation of cells in a matrix confers increased resistance to the deleterious compounds. For example, fermentation of plant biomass hydrolysate to produce biofuels is often complicated by the presence of several different compounds that are detrimental to the fermentation process. Encapsulation of microbial cells in a polymer matrix, calcium alginate for example, confers increased resistance to the compounds and thus improves the fermentative process.

The present patent document teaches new and improved processes and apparatuses for producing an encapsulated cell product. Previously, processes for producing encapsulated cell products were tailored for a specific end use, or were performed on a relatively small scale, or both. The present patent document teaches novel processes that increase the capacity of production of encapsulated cell products.

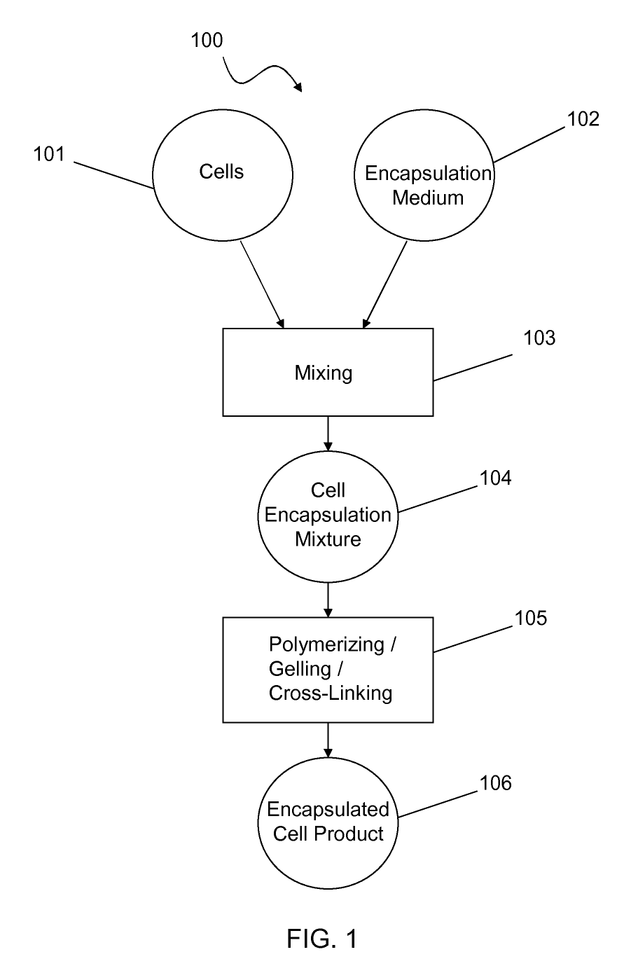

FIG. 1 illustrates a general process for the production of an encapsulated cell product 106. In the embodiment of process 100 shown in FIG. 1, cells 101 are combined with an encapsulation medium 102 in mixing step 103, to produce a cell encapsulation mixture 104. Cells 101 are preferably provided in a suspension that has been prepared for encapsulation. A solution of encapsulation medium 102 is mixed together with the suspended cells 101 to produce the cell encapsulation mixture 104. Once the cell encapsulation mixture is produced, it is polymerized, gelled or cross-linked in step 105 to produce an encapsulated cell product 106.

The cells 101 may be bacterial, yeast, fungal, algal, insect, or mammalian cells, or any other cell type as explained above. Cells 101 may also comprise a combination of such cells. The cells 101 may also be part of a functional group of cells or tissue, or be a product of cell fusion such as a hybridoma cell. The cells 101 may also be a mixture of cells growing symbiotically, for example a mixture of algal cells or a mixture of yeast or fungal cells. The type, combination, and density of the cells 101 are dependent on the desired use of the final encapsulated cell product 106.

The cells 101 may be provided to the process 100 in a number of different forms depending on the desired encapsulated cell product 106 to be created. If an encapsulated cell product 106 with a high cell density is desired, preferably the cells 101 are provided in a concentrated cell suspension. When the cells 101 are in a concentrated cell suspension, the mixing step 103 may be performed so as to maintain the high cell density in the resulting encapsulated cell product 106. A high concentration of cells in the encapsulated cell product 106 is advantageous for many biochemical processes, including, for example, fermentation applications. For example, a high concentration of yeast or bacterial cells may be required to produce a high fermentation rate in a reactor.

In other embodiments, the cells 101 may be in a relatively dilute suspension. The cells 101 may be provided in a dilute suspension so that the mixing step 103 and polymerizing step 105 are capable of encapsulating a single cell in an encapsulated cell product 106. Other combinations of cell suspensions 101 and mixing step 103 may be combined to provide different variations of encapsulated cell product 106.

In other embodiments, the cells 101 may be a functional group of cells or a tissue, such as a single human acinus from a human pancreas. Cells 101 could also include gap junctionally-connected or otherwise functionally-connected groups of neuronal and glial cells. The tissue may be provided in a dilute suspension so that the mixing step 103 and polymerizing step 105 are capable of encapsulating a single section of a tissue, such as a single acinus in an encapsulated cell product 106.

The encapsulation medium 102 may be a number of different materials. For example, the cells may be encapsulated using calcium alginate, a natural product of brown algae (seaweed). Other materials both natural and synthetic may be used including polymeric compounds such as sodium alginate, carrageenan, xantham gums, agarose, agar, cellulose and its derivatives, collagen, hyaluronate, pectin, gelatin, epoxy resin, photo cross-linkable resins, poly(vinyl alcohol), polyacrylamide, polyester, polystyrene, poly(vinyl acetate) and polyurethane to name a few. In some embodiments, more than one encapsulation medium 102 may be combined. For example, alginate and poly(vinyl alcohol) may be combined as the encapsulation medium 102.

Once both an aqueous solution of the encapsulation medium 102 and a concentrated cell suspension 101 are prepared, they are mixed 103 together in a vessel. Mixing step 103 may be accomplished using any method that produces the desired cell encapsulation mixture 104. It is important to accomplish mixing step 103 with minimal damage to the cells 101 and encapsulation medium 102. To this end, mixing step 103 should minimize the shearing forces on the cells 101 and encapsulation medium 102. In addition, the encapsulation medium 102 may be highly viscous, further complicating mixing. In exemplary embodiments, mixing step 103 is performed using a reciprocating shaker, or a reciprocating disk to mix cells 101 with the encapsulation medium 102. Standard impeller mixers will impart shear stress on the cells 101 and are not appropriate for mixing in highly viscous solutions.

Mixing step 103 also preferably mixes the cells 101 and encapsulation medium 102 to dispense the cells 101 throughout the encapsulation medium and achieve a preferred viscosity. In some embodiments, it may be desirable to mix until a substantially uniform dispersion is achieved. However, in other embodiments, the cells 101 and encapsulation medium 102 may not be mixed to homogeneity.

Once the cells 101 and the encapsulation medium have been mixed to a desired amount, preferably to homogeneity, the cell encapsulation mixture 104 may be polymerized, gelled, or cross-linked in step 105 to generate an encapsulated cell product 106. Prior to polymerizing step 105, the cell encapsulation mixture 104 may be formed into any number of structurally different products. For example, the cell encapsulation mixture 104 may be fashioned into spherical beads or threads, applied to a support structure such as a open mesh, honeycomb, or luffa, applied to reactor walls, or fashioned into another three dimensional shape before gelling, crosslinking or polymerizing 105 to produce the final product 106.

The nature of the polymerizing, gelling or cross-linking agent is specific to the polymers used as the encapsulation agent. In the case of sodium alginate, crosslinking of the alginate polymers is achieved by contact with different divalent or trivalent cations.

There are many techniques for improving the efficiency of an encapsulated cell product 106. One way to improve the efficiency is by providing a high surface area to volume ratio. Polymerizing the cell encapsulation mixture 104 increases its exposed surface area and may, therefore, increase the efficiency of the encapsulated cell product 106. For example, to aid in a biochemical process, such as a fermentation process, yeast cells in a sodium alginate solution may be polymerized into the form of small spherical beads, which provide a high surface area to volume ratio. Alternatively, yeast cells in a sodium alginate solution may be polymerized in the form of thin filaments or threads.

The cell encapsulation mixture 104 may be formed into beads using a drop-forming procedure. The resultant beads may be of different size and possess different pore sizes. There are many devices to produce beads. One may produce beads from a continuous stream by using electrostatic attraction to produce droplets, using vibration to produce droplets, using air to produce droplets, and using a rotating disk atomizer, to name a few. For example, if the matrix is sodium alginate, the beads are easily polymerized by contacting the beads with a calcium chloride solution.

In other embodiments, the cell encapsulation mixture 104 may be formed into thin filaments or threads. In this case, the filaments or threads can be slightly larger than the diameter of the cells entrapped. The filaments or threads may be deposited randomly to form a porous structure that may be used in a bioreactor. There are many means by which to produce thin filaments or threads, such as by extrusion through a narrow gauge needle followed by contact with a polymerizing agent or by electro-spinning. In the case of extrusion, a suspension of cells in an alginate matrix solution can be extruded under pressure through one, or, preferably, an array of narrow gauge hollow pins into a solution of calcium chloride to produce a large mass of filaments or threads.

In another embodiment, the cell encapsulation mixture 104 may be applied as a coating to a natural or synthetic, high surface area, support structure. In one implementation of this embodiment, the support structure only needs to be able to support the cell encapsulation mixture 104 and itself. For example, the support structure may comprise a ceramic sponge, honeycomb, reactor packing material or other support structure that increases the surface area per mass of the cell encapsulation mixture 104 when it is applied. In yet another embodiment, the cell encapsulation mixture 104 may also, or in the alternative, be applied to parts of the reactor surfaces, such as, the walls or the surface of the mixing devices.

An important aspect of the final encapsulated cell product 106 is that the resulting polymerized matrix is insoluble in aqueous medium or in the medium in which the product is to be used. In some cases, chemical agents contained in the medium may weaken or disrupt the polymers or the cross-linking agents in the encapsulated cell product 106. For example, several anions, such as citrate, phosphate, and sulfate may chelate calcium ions from calcium alginate, thus eliminating cross-linking and rendering the alginate soluble. Because of this, citrate, phosphate, and sulfate anions should be in low concentration or eliminated from media that is contacted with a calcium alginate encapsulated cell product.

Another important aspect of the encapsulated cell product 106 is that the resulting polymerized matrix contains pores which retain the encapsulated cells, but are permeable to different molecules that are required by the cell for maintenance or fermentation. For example, in the case of encapsulation of yeast in calcium alginate beads for fermentation of sugars to ethanol, the pore size of the encapsulated cell product 106 must be small enough to retain the yeast cells while allowing for the unrestricted movement of sugars into the bead and ethanol out.

Cells 101 may be encapsulated in an encapsulation medium 102 using a number of methods. All methods of cell encapsulation involve a mixing step 103, where cells 101 are mixed with the encapsulation medium 102 in a liquid form, followed by a gelling, cross-linking, or polymerization step 105, which completes the encapsulation of cells in the encapsulation matrix.

Alginate is ideal for use as an encapsulation medium 102. Alginate salts are soluble in aqueous media above pH 4, but are converted to alginic acid when the pH is lowered below about pH 4. A water-insoluble alginate gel is formed in the presence of gel-forming ions, e.g. calcium, barium, strontium, zinc, copper (II), aluminum, and mixtures thereof, at appropriate concentrations. Alginate gels are hydrogels, i.e. cross-linked alginate polymers that contain large amounts of water without dissolution. These properties make alginate gels ideal as an encapsulation medium 102.

Many polymer matrices originate as dry solids and are generally suspended in an aqueous solution prior to use. Some polymers may require suspension in an organic solvent before use. Because the degree of polymerization, gelling, or cross-linking is a function of the polymer length and concentration in solution, some polymers are used more routinely as an encapsulation matrix than others. Other considerations for a choice of polymer include: the relative ease of gelling, polymerization, or cross-linking the matrix to produce a final product; and the relative cell toxicity of the polymers themselves and of the cross-linking or polymerizing agent(s). More considerations include thermo-stability of the matrix in the specific application. Still more considerations include ease of use and the cost and commercial availability.

For many of the above stated reasons, alginates are commonly used for cellular encapsulation. Alginates are hydrophilic marine biopolymers with the ability to form heat-stable gels that can develop and set at low and moderate temperatures. Alginates are a family of non-branched polymers β-D-mannuronic acid and α-L-guluronic acid residues linked by 1-4 glycosidic bonds. Alginic acid is substantially insoluble in water, but forms water-soluble salts with alkali metals, such as sodium, potassium and lithium. Preparations of sodium alginate are commercially available.

In preparing sodium alginate for use as an encapsulation medium, a number of factors should be considered. For example, chain length, viscosity and concentration may all affect the effectiveness of the final encapsulated product. In addition, different sodium alginate products have different ratios of mannuronic acid and guluronic acid which occur naturally in different alginates. Alginates with specific mannuronic acid and guluronic acid ratios may be desirable for specific applications.

As a non-limiting example of process 100 in use to ferment sugars into ethanol, a dense slurry of Saccharomyces cerevisiae yeast cells may be used as the cells 101 and a solution of sodium alginate, a natural polymer from brown algae, may be used for the encapsulation medium 102. In other embodiments, other yeast cells may be used such as yeast from the genera Candida, Kluyveromyces, Pachysolen, Pichia, Saccharomyces, or others. When yeasts are mixed with the encapsulation medium 102, the concentration of yeast cells is preferably about 10 to 200 grams wet mass of cells per liter or about 25% wet mass of cells per liter after concentration.

Preferably, sodium alginate is mixed to a concentration of 1 to 6 grams per liter in the encapsulation medium 102. In one embodiment, the concentration of sodium alginate may be less than 3% w/v in water. The yeast slurry and the alginate solution are preferably mixed together to homogeneity in process step 103 to form a cell encapsulation mixture 104. Preferably, when the encapsulation medium 102 is sodium alginate, the viscosity of the encapsulation medium 102 is about 1500 to 3500 cSt and more preferably 2000 to 3000 cSt. The viscosity of the cell encapsulated mixture 104 is preferably 1000 to 3500 cSt and more preferably 1500-2500 cSt. Once the cell encapsulation mixture 104 is created, it may be polymerized 105. In one embodiment, polymerization is accomplished by passing the cell encapsulation mixture 104 through a 96-hollow pin device to produce droplets that are dropped into a solution of calcium chloride.

In another embodiment, the cells mixed with the encapsulation medium are bacterial cells, including the bacteria Escherichia coli, Zymomonas mobilis, or others. When bacteria are mixed with the matrix solution, the concentration of bacterial cells is preferably about 80 to 625 grams wet mass of cells per liter.

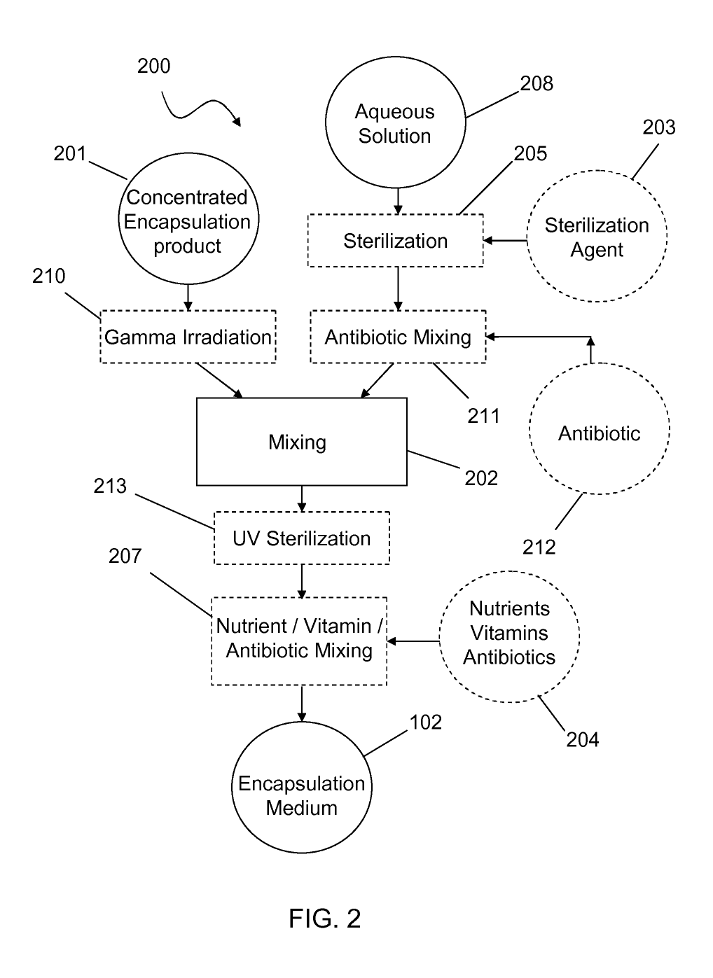

FIG. 2 illustrates a process for production of encapsulation medium 102 from a concentrated encapsulation product 201. The concentrated encapsulation product 201 may begin in the solid or liquid state and may have the full range of viscosities in between. In one embodiment, the concentrated encapsulation product 201 is a dry powder. Other embodiments of the process may begin with a polymer or other medium that is in liquid form. Storing the concentrated encapsulation product 201 in a dry powder form, or as concentrated and dehydrated as possible, is advantageous because substances typically have an increased shelf life in the dry form. Furthermore, the dry concentrated form reduces the storage volume and allows easier and cheaper transportation. However in other embodiments, the process shown in FIG. 2 may begin with a concentrated encapsulation product 201 in any viscous state from solid to liquid.

In the embodiment shown in FIG. 2, a concentrated encapsulation product 201 is provided and solubilized with an aqueous solution 208 in mixing step 202. The mixing step creates a solubilized encapsulation medium which may function as an encapsulation medium 102. However, in other embodiments, the solubilized solution may be further processed by other optional steps as explained below. Once the encapsulation medium 102 is created, it may be mixed with cells 101 as shown in FIG. 1

In a preferred embodiment, the concentrated encapsulation product 201 is a polymer. More preferably, the concentrated encapsulation product 201 may be sodium alginate. Sodium alginate is available in various forms from a number of sources including: WEGO Chemical and Mineral Co., 239 Great Neck Road Great Neck, N.Y. 11021 (www.wegochem.com); Sigma-Aldrich, 3050 Spruce St. St. Louis, Mo. 63103 (www.sigmaaldrich.com); and MP Biomedicals, 29525 Fountain Pkwy. Solon, Ohio 44139 (www.mpbio.com).

The embodiment shown in FIG. 2 illustrates adding an aqueous solution 208 to solubilize the concentrated encapsulation product 201 in mixing step 202. In one embodiment of the process 200, the concentrated encapsulation product 201 is introduced in a dry state into a mixing vessel containing a liquid by use of a mixing eductor. In the preferred embodiment, the aqueous solution is water. However, in other embodiments the aqueous solution may be a combination of water and other additives that may enhance the performance of the final encapsulation medium 102.

Mixing step 202 is performed to produce an encapsulation medium 102 with the desired concentration. In an exemplary embodiment of process 200, sodium alginate powder is used as the concentrated encapsulation medium 201. The sodium alginate powder may be added to the aqueous solution 208 to a concentration of about 1% w/v to 6% w/v. Preferably, the sodium alginate used is a granulated product of about 320 mPa to 1400 mPa, where the viscosity of aqueous sodium alginate solution used for the encapsulation is 1500 to 3500 centistokes (cSt). A granulated sodium alginate is preferred because a granulated product is solubilized more easily in water between about 15 to 30° C.

The concentrated encapsulation product 201 may contain microbial contaminants such as bacteria. To reduce the level of contamination of the concentrated encapsulation product 201, a number of different sterilization procedures 205, 210 and 213 are optionally possible by different embodiments of process 200. In addition, other embodiments may have antibiotics added 207 and 211 before or after mixing 202.

In one embodiment of the process 200, optional gamma irradiation 210 may be used to sterilize the concentrated encapsulation medium 201. Gamma irradiation may be used at 8 to 20 kilogray (kGy) to irradiate the concentrated encapsulation medium.

In other embodiments, the aqueous solution 208, which is sterile, may be used as a vehicle to sterilize the concentrated encapsulation medium 201 during the mixing step 202. The aqueous solution 208 may have a chemical sterilization agent 203 or antibiotic 212 added prior to mixing 202 with the concentrated encapsulation product 201 to reduce the level of microbial contamination in the resulting mixture. In such an embodiment, a sterilizing agent 203 is mixed 205 with the aqueous solution 208 prior to the mixing step 202. In certain embodiments, antibiotics 212 may also be added to the aqueous solution 208 prior to the mixing step 202. The aqueous solution 208 distributes the sterilization agent 203 and/or antibiotic 212 into the concentrated encapsulation medium 201 during the mixing step 202. By first mixing the sterilization agent 203 and/or antibiotics 212 with the aqueous solution 208, the sterilization agent 203 and/or antibiotics 212 may be more effectively evenly distributed to the concentrated encapsulation medium 201.

In other embodiments of process 200, the encapsulation medium may be sterilized after mixing 202 by using ultraviolet irradiation 213 to reduce the microbial contamination of the final mixture 102.

Although immediate use of the encapsulation medium is always preferable, optional sterilization steps 205, 210 and 213 may be desirable to increase the shelf life of the solubilized encapsulation medium for storage. In addition, certain embodiments may add antibiotics either before or after the mixing step 202. Because concentrated encapsulation products are often not sterile and their shelf life after solubilization is shortened by growth of undesirable microorganisms, it may be desirable to treat the concentrated encapsulation medium with some of the optional steps described. Furthermore, because the growth of unwanted organisms in the solution may decrease the viscosity of the encapsulation medium 102, various sterilization steps may help prevent unwanted changes in viscosity.

Although the optional sterilization steps 205, 210 and 213 were described separately above, in various embodiments the sterilization steps may be used in any combination. In addition, the sterilization step 205, 210 and 213 may be used in any combination with the optional addition of antibiotics.

The present patent document teaches the novel idea of using non-thermal sterilization methods 205 and mixing methods 202. In order to get some concentrated encapsulation products 201 into solution, the mixture may be heated and stirred on a stir plate or, more commonly, heated in a laboratory autoclave at 121° C. for 15 to 45 minutes. However, heating alginate polymers may cause some amount of hydrolysis of the alginate and thereby change the properties of the alginate solution, including its viscosity.

Thermal methods are also often used to sterilize the solution. If mixing step 202 and sterilization steps 205, 210 and 213 are performed using a non-thermal method, a substantially lower concentration of concentrated encapsulation product 201 is needed in the final encapsulation medium. For example, if sterilization step 205 is performed using a thermal method such as autoclaving, a sodium alginate concentration of around 3.5% (w/v) sodium alginate to water is required to maximize the ethanol yield of the resulting encapsulated cell product. However, if mixing step 202 and sterilization steps 205, 210 and 213 use a non-thermal method such as chemical sterilization, less than 3% sodium alginate and preferably less than 2.5% and more preferably about 2% sodium alginate (w/v) sodium alginate to water is needed to maximize the ethanol yield of the resulting encapsulated product. Despite the lower concentration of sodium alginate in the final encapsulated cell product, the performance of the encapsulated cell product is not decreased.

By reducing the concentration of encapsulation product 201 that is required to produce encapsulated medium 102, without affecting the yield of the encapsulated cell product 106, performing sterilization steps 205, 210 and 213 using a non-thermal method provides a substantial cost savings over thermal methods. In particular, when the process of FIG. 2 is scaled up to an industrial scale, the reduction in the concentration of encapsulation product 201 needed to create encapsulation medium 102 may provide significant cost savings.

Various chemical sterilization products may be used as sterilization agent 203. For example, in certain embodiments sterilization agent 203 may be selected from the group consisting of Lactrol (by PhibroChem), Lactoside (by Lallemand Ethanol Technologies); FermaSure (by DuPont); FermGuard (by Ferm Solutions); FermGuard Xtreme (by Ferm Solutions); sodium chlorite; and Chloramphenicol. In addition, more than one sterilization agent 203 may be combined together.

In an embodiment that uses sodium chlorite as a sterilization agent 203, the concentration of the sodium chlorite may be about 1 to 2000 parts per million (ppm). The sodium chlorite solution may be the commercial product FermaSure.

In another embodiment designed to retain sterility in the encapsulation medium 102, the antibiotics penicillin and virginiamycin may be added after sterilization. Preferably, the commercial product Lactoside may be added at a concentration of about 1 to 5 ppm. In other embodiments, other antibiotics may be used including industrially produced antibiotics such as FermGuard Xtreme in concentrations of 1 to 5 ppm.

As explained above, rather than adding a sterilization agent 203 or in addition to adding a sterilization agent 203, certain embodiments of process 200 may sterilize the concentrated encapsulation medium 201 using ultraviolet irradiation 213. Preferably, the encapsulated medium mixture 102 is irradiated with ultraviolet light at 10 to 50 mWs/cm² or 10 to 50 mJ per cm² to achieve sterilization.

In addition to optional sterilization 205 and optionally mixing in antibiotics, the encapsulation medium 102 may further be processed in a nutrient/vitamin mixing step 207. Vitamins and nutrients 204 may be added into the encapsulation medium 102 in preparation for mixing with the cells 101 as shown in FIG. 1. By creating an encapsulation medium 102 that already contains vitamins and nutrients essential to cell growth, the cells 101 may become encapsulated in an environment that enhances growth, production and efficiency of the cells 101.

In one embodiment, the vitamins and nutrients 204 may be corn steep powder or corn steep liquor. The corn steep powder may be used at a concentration of 1 to 5 percent weight/volume. The corn steep liquor may be used at a concentration of 1 to 5 percent volume/volume.

In other embodiments, individual vitamins or a mixture of vitamins may also be added. In one embodiment, the vitamin biotin may be added in the mixing step at a concentration of 2 ng/L to 2 micrograms/L. In another embodiment, the vitamins biotin and thiamine may be added in the mixing step at a concentration of 4 ng per liter to 400 micrograms per liter.

FIG. 2 shows sterilization 205, antibiotic mixing 211, and nutrient/vitamin mixing 207 as optional additional steps. However, in other embodiments, sterilization 205, antibiotic mixing 207 and nutrient/vitamin mixing 207 may be performed in a single mixing step 202 and/or in the same mixing vessel. For example, the concentrated encapsulation product may be dissolved into growth medium, into a mixture of vitamins including biotin and or thiamine, or into growth medium supplemented with vitamins, or into a natural solution containing biotin and or thiamine. The mixing step 202 may be performed in the same vessel or in separate vessels.

The process 200 for making an encapsulation medium 102 and the process 100 for mixing the encapsulation medium 102 with cells 101 to make an encapsulated cell product 104 both require mixing step 202 and 103 respectively. In a preferred embodiment, mixing step 202 and 103 may happen in the same vessel. Preferably, the mixing 103 of the encapsulation medium 102 and cells 101 occurs in the same disposable vessel in which the encapsulation polymer is solubilized and sterilized.

The following example will be referred to as Example 1 and demonstrates the application of one embodiment of the process of FIG. 2 for producing an encapsulation medium 102 from sodium alginate. In Example 1, the process shown in FIG. 2 was used to produce five (5) different batches of an encapsulation medium. Each of the five (5) different batches started with a different sodium alginate product as the concentrated encapsulation medium 201. Each of the five (5) sodium alginate products had a different polymer length. The viscosity of the sodium alginate is proportional to the polymer length and accordingly, Example 1 is provided to illustrate the effect of varying the viscosity of the concentrated encapsulation product 201 (such as sodium alginate) on the ethanol producing efficiency of the polymerized encapsulated cell product 106. As shown by Example 1, varying the viscosity of the concentrated encapsulation product can affect the ethanol producing efficiency of an encapsulated microbe in an ethanol producing bioreactor.

In Example 1, all of the varying batches were mixed with Zymomonas mobilis 8b as shown in FIG. 1, polymerized into beads, and used to ferment sugar cane bagasse hydrolysate. Each of the five (5) batches used a different sodium alginate product with a different viscosity as the concentrated encapsulation product 201. The sodium alginate products with vary viscosities were then solubilized by mixing them with water in mixing step 202 by adding 3.5% (weight (w)/volume (v)) sodium alginate to water. In all cases, the biomass loading of the Zymomonas was 3% and the inoculation was 0.2 g beads per ml hydrolysate solution. The hydrolysate solution was supplemented with 1.2 g/L diammonium phosphate and 0.5 w/v yeast extract. Incubations were at 30° C. for 71 hours at 50 rpm on a rotary shaker.

Table 1 shows the ethanol concentrations following incubation of Zymomonas mobilis 8b encapsulated in 5 different alginate products in sugar cane bagasse hydrolysate. The data is shown as the average of triplicate determinations. Viscosities for the different sodium alginate products (1% in 1% acetic acid at 20° C.) range from 100 or 200 mPa, to even as much as 1236 mPa. The data shows that the two alginates that resulted in the highest ethanol concentrations were the 324 mPa and 620 mPa sodium alginate products produced by Wego Chemical and Mineral Co. Accordingly, a preferred embodiment for use fermenting sugar cane bagasse hydrolysate, includes alginate with a medium to low viscosity of about 324 mPa to 620 mPa.

TABLE 1
Final ethanol concentration as a function of alginate viscosity.

| Alginate | Ethanol (g/L) |
|---|---|
| Sigma chemical | 13.6 |
| MP BIO | 14.5 |
| Wego (324 mPa) | 15.6 |
| Wego (620 mPa) | 16.9 |
| Wego (1236 mPa) | 14.2 |

Varying the viscosity of the concentrated encapsulation product may be advantageous for a number of reasons. For example, if the beads are to be used in a solid-state reactor, a harder bead may be desirable to better maintain its shape under the weight of the biomass and other beads. Accordingly, different viscosities may be desired depending on the purpose for which the encapsulation medium is being produced. Factors that will affect the desired viscosity of the encapsulation medium include but are not limited to the type of hydrolysate being fermented, the cell being encapsulated and the type of reactor the encapsulation medium will eventually end up in.

A second example, Example 2, of one embodiment of the process of FIG. 2 for producing an encapsulation medium 102 from sodium alginate will now be discussed. Example 2 is provided to illustrate the effect of varying the concentration of the sodium alginate (or other encapsulation medium) on the ethanol efficiency of the polymerized encapsulated cell product. As shown by Example 2, varying the concentration of the sodium alginate in the encapsulation medium affects the ethanol producing efficiency of an encapsulated microbe in an ethanol producing bioreactor.

In Example 2, the process shown in FIG. 2 was used to produce five (5) different batches of an encapsulation medium starting with the same sodium alginate product but varying the concentration of sodium alginate in the final encapsulation medium. The varying batches were then each mixed with Zymomonas mobilis 8b as shown in FIG. 1, polymerized into beads, and used to ferment sugar cane bagasse hydrolysate.

The final concentration of sodium alginate, or other encapsulation product, will depend on the volume of solubilizing agent added and also on the volume of concentrated cell slurry and other additives such as sterilization chemicals and vitamins and nutrients that are present in the final encapsulated cell product 106.

In Example 2, each of the five batches used the same sodium alginate as the concentrated encapsulation product 201. The sodium alginate was then solubilized by mixing it with varying amounts of water from 0.05% to 10% (weight (w)/volume (v)) sodium alginate to water. In all cases, the biomass loading of the Zymomonas was 3% and the inoculation was 0.2 gram beads per milliliter hydrolysate solution. Incubations were at 30° C. for 48 hours at 80 rpm on a rotary shaker. The data is shown as the average of triplicate determinations.

Table 2 shows the ethanol concentrations following incubation of Zymomonas mobilis 8b, encapsulated in 5 different concentrations of Wego 324 mPa sodium alginate, in sugar cane bagasse hydrolysate. The data is shown as the average of triplicate determinations. The data shows that the percent alginate that resulted in the highest final ethanol concentration after fermentation was about 2% (w/v) sodium alginate to water or about 2 grams sodium alginate per 100 ml of aqueous solution. Accordingly, a preferred embodiment for use fermenting sugar cane bagasse hydrolysate, includes mixing sodium alginate to a concentration of about 1%-2% (w/v) sodium alginate to water and more preferably about 1.75%-2% (w/v) sodium alginate to water and even yet more preferably 2% (w/v) sodium alginate to water.

TABLE 2
Final ethanol concentration as a function of alginate concentration.

| Alginate concentration | Ethanol |
|---|---|
| (% w/v) | (g/L) |
| 1 | 4.5 |
| 1.5 | 4.4 |
| 1.75 | 4.5 |
| 2 | 4.7 |
| 3.5 | 4.3 |

In addition to performing the process shown in FIG. 2 to create an encapsulation medium 102 that has the desired initial viscosity, it is also important to maintain the viscosity of the encapsulation medium 102 if it is not immediately used. As mentioned above, the viscosity of the encapsulation medium 102 may be reduced by the growth of unwanted organisms. Consequently, it may be advantageous to perform some of the optional sterilization steps either individually or in combination if the encapsulation medium will be stored for any length of time prior to use.

A third example, Example 3, of one embodiment of the process of FIG. 2 will now be discussed. Example 3 shows the beneficial effects of including optional sterilization step 205 in the process of FIG. 2 if the encapsulation medium 102 is to be stored prior to use.

In Example 3, six (6) separate batches of 2% w/v solutions of sodium alginate (Wego 324 mPa) were prepared as encapsulation mediums 102. One of the batches was not sterilized at all and each of the other five (5) batches was sterilized 205 with a different sterilization agent 203. The sterilization agents 203 were selected from the group including: Lactoside; FermaSure; FermGuard; FermGuard Xtreme; and chloramphenicol. Each of the 6 batches was then allowed to incubate for 120 hours at room temperature. The subsequent viscosities after incubation were determined using a Zahn Cup-type viscometer. The data shows that the use of a commercial antibiotic that is generally recognized as safe (GRAS) aids in keeping the viscosity of the alginate solution high.

TABLE 3
Alginate viscosities after antimicrobial treatment.

| | Concentration | |
|---|---|---|
| Treatment | (ppm) | Viscosity (cSt) |
| None | — | 37 |
| Lactoside | 2 | 463 |
| FermaSure | 15 | 482 |
| FermGuard | 2 | 449 |
| FermGuard Xtreme | 2 | 36 |
| Chloramphenicol | 50 | 605 |

Cells are generally prepared in some method before encapsulation in an encapsulation matrix. One preparation is to prepare a certain density of cells for encapsulation. Depending on the final use of the matrix encapsulated cell product 106, the cells encapsulated in a matrix may either be in a high density or a low density. For example, in fermentation processes to produce organic acids, antibiotics, or ethanol, it is desirable for cells to be in a high density in the ‘free’state in the fermentation reactor, it would therefore also be advantageous for cells to be at high density in the encapsulation matrix.

In order to achieve a high cell density in a fermentation reactor, for example, cells must first be concentrated to a high density before mixing with an encapsulation matrix. A common laboratory method to achieve high cell density as a means of preparing a cell concentrate for encapsulation into an encapsulation matrix is to centrifuge a cell culture to increase the cell concentration. The concentrated cell suspension is then mixed with the encapsulation medium. Although continuous centrifuges and large batch centrifuges are available in industry, both centrifugation systems are unsuitable for production of very large quantities of concentrated cells in short time periods.

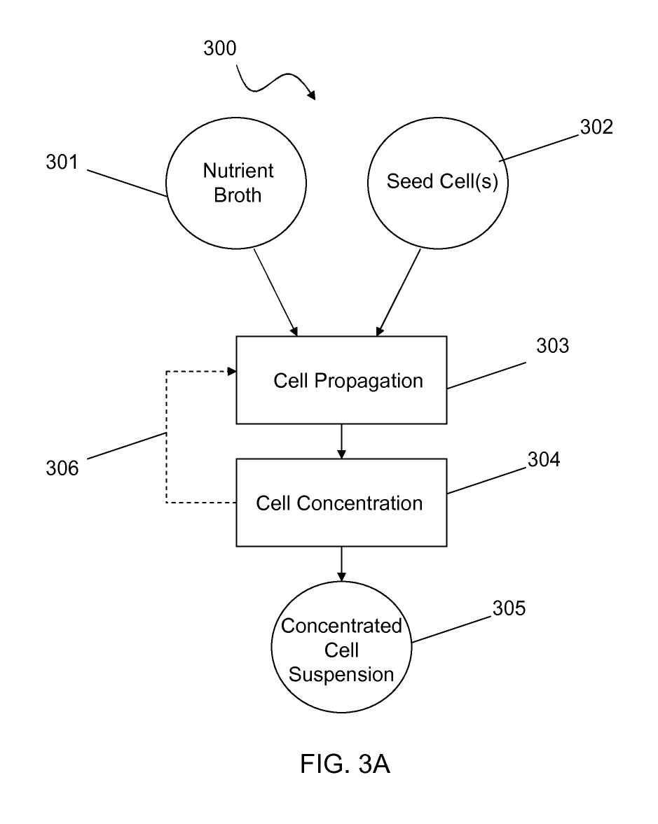

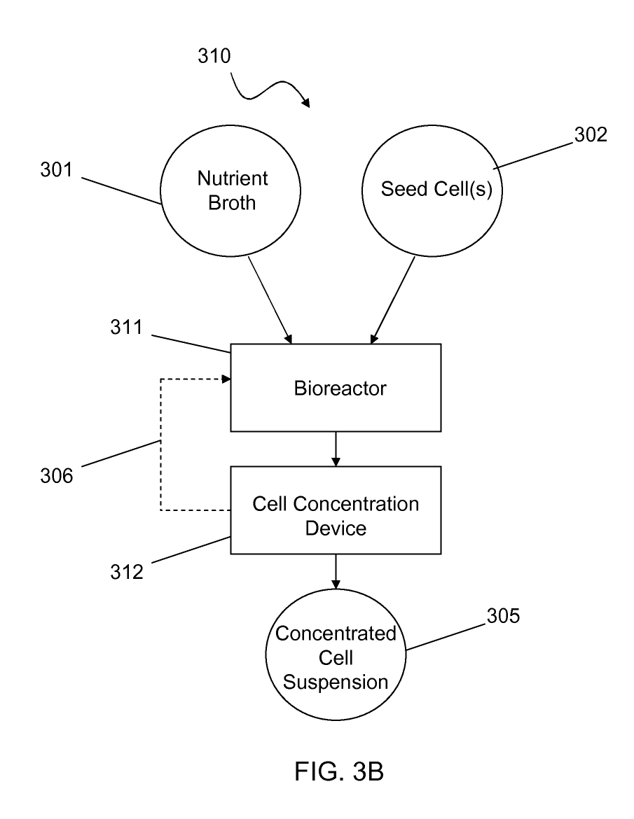

FIG. 3A illustrates an embodiment of a process 300 for producing a concentrated cell suspension 305. In one embodiment, the process 300 may be used to create a concentrated cell suspension 305 for use as the cells 101 in process 100 of FIG. 1. As shown in FIG. 3, a seed cell or cells 302 is combined with a nutrient broth 301 and the cells 302 are allowed to propagate throughout the nutrient broth 301. The cells are then concentrated 304 into a concentrated cell suspension 305.

FIG. 3B illustrates an embodiment of a system for production of a concentrated cell suspension as shown in FIG. 3A. The cell propagation is preferably performed in a bioreactor 311. The process 300 may be a batch process or a continuous process and the type of bioreactor 311 may be selected accordingly. In a batch process, the bioreactor 311 is preferably a type of bioreactor designed for batch processing such as a packed bed reactor or a stirred tank reactor. In a preferred embodiment of process 300 using a batch mode of operation, propagation 303 of a subsequent batch of cells initiates while the previous batch is being concentrated 304.

Other embodiments of process 300 may use a continuous process instead of a batch process. In a preferred embodiment of a continuous process 300, bioreactor 311 is a chemostat bioreactor, however, other types of continuous reactors may be used. In embodiments of process 300 that are continuous, fresh growth medium is continuously added, while medium containing cells is continuously removed. Thus, under continuous processing conditions, a continuous supply of cell suspension is produced for ultimate encapsulation.

Numerous types of bioreactors are marketed by various companies including: Xcellerex, Inc., 170 Locke Drive Marlborough, Mass. 01752-7217; Sartoius AG, Weender Landstrasse 94-108, D-37075 Goettingen, Germany; Thermo-Scientific (HyClone) 925 West 1800 South Logan, Utah 84321; and Millipore, 290 Concord Road, Billerica, Mass. 01821.

In order to create a large cell suspension for use in industrial or other large scale applications, the bioreactor 311 used to propagate the cells may also be large in size. For example, cells may be propagated in at least a 10 liter (L), or more preferably at least a 200 L, or even more preferably at least a 2000 L bioreactor 311.

The concentration of the cells in the bioreactor 311 after cell propagation 303 will vary depending on many factors such as the cell type, the nutrient broth, and the type of bioreactor. Typically cell concentrations after cell propagation 303 range from between about 1 and about 25 grams wet mass per liter growth medium. There are numerous applications, such as the fermentation of biomass, that are more effective if the density of the cells can be further increased.

In the embodiments shown in FIGS. 3A and 3B, the cells are further concentrated in cell concentration step 304 in a cell concentration device 312. It is important that the cell concentration step 304 does not significantly affect the viability of the cells 302.

In a preferred embodiment, the cell concentration step 304 is performed by concentrating the cells on a membrane and harvesting them. In such a preferred embodiment, the cell concentration device 312 is preferably a tangential flow filtration system.

In a tangential flow filtration system, also known as a cross flow filtration system, the feed is passed across a filter membrane (tangentially) at positive pressure relative to the permeate side. A proportion of the material which is smaller than the membrane pore size passes through the membrane as permeate or filtrate; everything else is retained on the feed side of the membrane as retentate.

A tangential flow filtration system separates cells from the growth medium and collects the cells on the membrane or filter. Cells may then be harvested from the membrane and collected. The tangential flow filtrations system can produce a cell slurry, while maintaining high cell viabilities.

In yet another embodiment of process 300, a concentrated cell suspension 305 is achieved by operationally integrating the cell propagation process 303 with the cell concentration process 304. In an integrated process 300, harvest of the concentrated cells in the tangential flow filtration retentate is used to produce the encapsulated cell product and may be used as the inoculum for serial propagation batches.

In a preferred system 310 design to run as an integrated system 300, the cell concentration device 312 may be operationally and functionally connected to the bioreactor 311 so that the cells may be easily transferred between the bioreactor 311 and the concentration device 312. Although, integrated propagation 303 and concentration 304 may be used for either batch or continuous processing, connecting the bioreactor 311 with the cell concentration device 312 is especially desirable when process 300 is designed as a continuous process. In other non-integrated embodiments, the bioreactor 311 and the concentration device 312 may not be connected and the cells 302 are externally transferred between the two devices.

In another embodiment, the cell concentration device 312 further includes a cell concentration holding vessel. The cell concentration holding vessel allows the cell concentration device 312 to repeatedly cycle the concentrated cells back through the concentration process and thereby further increase the concentration of the cells. As an exemplary embodiment, a tangential flow filtration system is connected to a retentate holding vessel. The cells are repeatedly cycled back through the tangential flow filtration system from the retentate holding vessel to repeatedly increase the cell concentration. In such an embodiment, the cells may be concentration to about 80 to 625 grams wet mass cells per liter.

In other embodiments, other cell concentration devices 312 may be used such as a centrifuge. Another means by which to produce concentrated cell suspensions is to rely on the natural flocculating behavior of some cells. In the case of flocculating yeast, for example, sedimented flocs settling to the bottom of a bioreactor contain a high cell concentration. These flocs may be harvested and mixed with an encapsulation medium or have encapsulation matrix added to them. One drawback to this cell concentration method is that only a low number of cell species floc or floc under conditions where valuable products are produced.

In other embodiments, free cells are allowed to sediment in a reactor vessel to increase the cell density. The sedimented cells may then be harvested at high density for subsequent mixing with an encapsulation medium. One drawback to this method is a loss of cell viability following sedimentation. Another drawback is that certain cells are motile and do not sediment easily.

In certain embodiments of process 300, cells may be propagated by growing the cells batch-wise in a bioreactor under conditions specific to the generation of cell biomass. Repeated propagation of the cells in a single bioreactor vessel may be accomplished without the benefit of sterilization of the bioreactor vessel between propagation batches in this process.

In embodiments of process 300 that are designed to be a batch process, a portion of the cells from the cell concentration step 304 may be reused in optional step 306 as an inoculum for the next batch of cell propagation. In a preferred embodiment where an inoculum is used, 2% to 20% and more preferably 5% to 10% of the cells from the cell concentration are recycled back from the cell concentration device 312 to the bioreactor 311 to act as an inoculum for the next batch of cell propagation 303. By using a portion of the prior batch of cells as an inoculum for a subsequent batch of cells, the bioreactor may process numerous batches of cells without any additional sterilization. In embodiments where an inoculum is used, fresh growth medium may be added to the cell inoculum by introduction through a sterile filter into the propagation bioreactor.

In embodiments of process 300 that are designed to be run as a continuous process, a portion of cells from a previous process may also be used an inoculum to get the continuous process started.

A tangential flow filtration device is the preferred embodiment of the cell concentration device 312. Concentrating the cells using a tangential flow filtration device is particularly well suited for the production of large volumes of calcium alginate beads having one or more fermentative cells encapsulated therein. Table 4 shows examples of tangential flow filtration to concentrate propagated fermentative cells, such as Pachysolen and NREL's Zymomonas mobilis 8b.

TABLE 4
Concentration of microbial cells by tangential flow filtration.

| | | | | Cell Mass Yields | | |
|---|---|---|---|---|---|---|
| | TFF Concentration | | | Estimated | Actual | |
| | Permeate | Retentate | | Propagation | Retentate | |
| Examples | Volume | Volume | Concentration | Wet Mass | Wet Mass | % Viable |
| Propagated Cell | (L) | (L) | Factor | (g/L) | (g/L) | Cells |
| Pachysolen sp. | 9.30 | 0.63 | 15 | 24.8 | 396.2 | 96.0% |
| Zymomonas mobilis 8b | 11.40 | 0.77 | 15 | 10.4 | 182.3 | 94.5% |
| (NREL) | | | | | | |
| Zymomonas mobilis 8b | 10.75 | 0.52 | 21 | 10.4 | 230.8 | >99% |
| (NREL) | | | | | | |
| Zymomonas mobilis 8b | 179.00 | 18.00 | 10 | 18.5 | 206.1 | >99% |
| (NREL) | | | | | | |
| Zymomonas mobilis 8b | 205.20 | 11.80 | 17 | 11.7 | 218.8 | >99% |
| (NREL) | | | | | | |

As shown in Table 4, cells can be concentrated between 10 and 21 times their normal propagation concentration by using a tangential flow filtration device. In other embodiments, cells may be concentrated even more than 21 times their normal propagation concentrations. Furthermore, using a tangential flow filtration system has very little effect on the overall viability of the cells. A high concentration of cells in the cell suspension is desirable to create an encapsulated cell product with a high cell concentration. Encapsulated cell products with high cell concentrations outperform lower cell concentrations in many situations including a fermentation reactor.

Following cell concentration 304, the concentrated cells 305 are recovered and maybe used in a process 100 as shown in FIG. 1 to produce an encapsulated cell product 106. As shown in FIG. 1, the concentrated cells are thoroughly mixed 103 with an encapsulation medium 102 such as sodium alginate. The desired cell concentration within the encapsulation medium is both organism and substrate dependent. For example, a suitable target loading for Pachysolen tannophilus in sodium alginate for fermenting hydrolysate is at least 5 g cells/100 mL sodium alginate medium.

Mixing 103 the encapsulation medium 102 with the concentrated cell suspension 305 may occur in the same device or vessel that is used for the suspension of the alginate or the mixing may occur in a separate device. For example, sodium alginate may be prepared in a mixing bag and then the cells 101 may be aseptically added to the same bag to form the cell encapsulated mixture 104.

Referring back to FIG. 1, mixing step 103 may mix more than one type of cell in the same encapsulation medium 102 so that the encapsulated cell product contains a plurality of types of cells with complimentary properties. For example, cells that are particularly good at fermenting five carbon sugars might be combined in the same encapsulated cell product with cells that are particularly good at fermenting six carbon sugars.

When combining cells with complimentary properties, the cells may be combined within the same encapsulation vehicle, or the cells may be encapsulated separately and the separately encapsulated cells combined in the same fermentation reactor. For example, if calcium alginate beads are used as the encapsulation vehicle, different complimentary cells may be combined within the same bead. As one example, to effectively ferment softwood hydrolysate, which contains the sugars mannose, galactose, glucose and xylose, to ethanol, one may combine Zymomonas mobilis, NREL strain 8b, which ferments glucose and xylose to ethanol, with Saccharomyces cerevisiae, which ferments mannose and galactose, into a single bead product. In this way advantageous fermentative properties of different microbial species are combined in a single bead product.

In the preferred embodiment of process 300, a disposable vessel is reused for individual cell propagation batches. One embodiment of a disposable vessel is a plastic polymer disposable bioreactor bag in a plastic or metal frame. The disposable vessel may preferably be used for between 1 and 10 individual cell propagation batches and more preferably be used for 3 to 4 cell propagation batches. In certain embodiments when a disposable vessel is reused for multiple cell propagation batches, the vessel is not sterilized between batches. In another embodiment, 5% to 10% of the cell biomass from the previous propagation batch may be used as an inoculum for the subsequent batches. If a tangential flow filtration system is used as the filtration device, a portion of the concentrated retentate may be used as the inoculum and transferred to the propagation bioreactor from the retentate holding vessel.

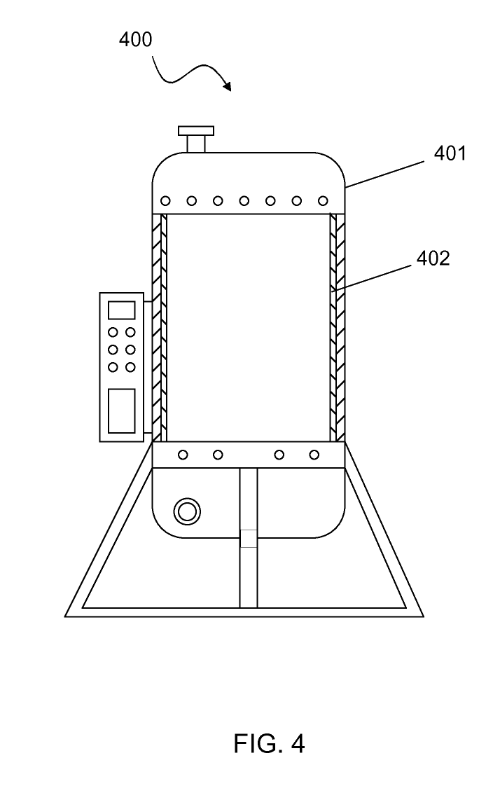

FIG. 4 illustrates an example of a bioreactor that uses a reusable and disposable bag. FIG. 4 is one embodiment of a bioreactor 400 that includes a frame 401 and a disposable and/or reusable bag 402. Bioreactor 400 is one embodiment of a bioreactor 311 that may be used in system 300 of FIG. 3.

Using a bioreactor 400 including a disposable bag 402 provides several advantages over other bioreactor designs. Disposable bags 402 obviate expensive Clean In Place (CIP) procedures and sanitization validation procedures that must be used when using stainless steel fermentation tanks. In addition, a disposable bag can help control contamination issues because it may be disposed of and replaced with a new sterile bag at any time. While the disposable bag 402 may be replaced with each batch, as explained above the disposable bag may also be used for a number of batches before being discarded. In a preferred embodiment of a process 300 including a bioreactor 311 including a disposable bag 402, the disposable bag 402 is used for 3 to 4 batches before being disposed of Reusing the disposable bag reduces the recurring cost of replacing the disposable bag 402.

Bioreactor 400 including reusable bag 402 may be used for both batch processing and continuous (chemostat) processing.

The processes and apparatuses described herein are designed to be particle even when scaled up to create an encapsulated cell product on an industrial scale. For example, the processes and apparatuses described herein can be scaled up to service industrial reactors or a series of industrial reactors. Industrial reactors may be on the scale 20,000 L (75,000 gal.) or even larger.

Although the embodiments have been described with reference to the drawings and specific examples, it will readily be appreciated by those skilled in the art that many modifications and adaptations of the processes and apparatuses described herein are possible without departure from the spirit and scope of the embodiments as claimed hereinafter. Thus, it is to be clearly understood that this description is made only by way of example and not as a limitation on the scope of the embodiments as claimed below.

What is claimed is:

1. A process for production of an encapsulated cell product, the process comprising the steps of:
a. concentrating cells from a propagation medium using a tangential flow filtration system;
b. mixing concentrated cells with an encapsulation medium to form a cell encapsulation mixture; and
c. polymerizing the cell encapsulation mixture to form an encapsulated cell product.

2. The process of claim 1, wherein the cells are concentrated in the retentate of the tangential flow filtration system.

3. The process of claim 1, further comprising the step of using retentate from the tangential flow filtration system as an inoculum for a subsequent production of an encapsulated cell product.

4. The process of claim 2, further including the step of cycling the retentate between a retentate holding vessel and the tangential flow filtration system.

5. The process of claim 4, wherein the cycling step is performed until the retentate achieves a cell concentration of greater than 180 grams wet mass of cells per liter.

6. The process of claim 1, wherein the mixing step is performed with a reciprocating shaker.

7. The process of claim 1, wherein the mixing step is performed with a reciprocating disk.

8. A system for production of an encapsulated cell product, the system comprising:
a bioreactor;
a tangential flow filtration system in communication with the bioreactor; and
a retentate holding vessel in communication with the tangential flow filtration system.

9. The system of claim 8, further comprising a reciprocating shaker operatively arranged to mix a cell suspension from the retentate holding vessel with an encapsulation medium.

10. The system of claim 8, wherein the bioreactor is a chemostat bioreactor.

11. A process for production of an encapsulated cell product, the process comprising the steps of:
a. concentrating cells from a propagation medium;
b. mixing the concentrated cells with an encapsulation medium using a reciprocating shaker to form a cell encapsulation mixture; and
c. polymerizing the cell encapsulation mixture to form an encapsulated cell product.

12. The process of claim 11, wherein the bioreactor is a chemostat bioreactor.

13. The process of claim 11, wherein the cells are concentrated to greater than 180 grams wet mass of cells per liter.

14. The process of claim 11, wherein the encapsulation medium has a viscosity of about 1000 to 3500 centistokes.

15. The process of claim 11, wherein the cell encapsulation mixture has a viscosity of about 1000 to 3500 centistokes.

16. A process for production of an encapsulated cell product, the process comprising the steps of:
a. propagating cells in a bioreactor to form a first batch of cells;
b. concentrating cells from the first batch of cells;
c. mixing concentrated cells with a encapsulation medium to form a cell encapsulation mixture;
d. polymerizing the cell encapsulation mixture to form an encapsulated cell product; and
e. propagating cells in a bioreactor to form a second batch of cells using cells from the first batch of cells as an inoculum.

17. The process of claim 16, wherein the bioreactor is not sterilized between the propagating the first batch of cells and propagating the second batch of cells.

18. The process of claim 16, wherein the concentrating step is performed by a tangential flow filtration system and the cells are concentrated in a retentate.

19. The process of claim 18, further comprising the step of using a portion of the retentate as the inoculum.

20. The process of claim 18, further including the step of cycling the retentate between a retentate holding vessel and the tangential flow filtration system.

21. The process of claim 20, wherein the cycling step is performed until the retentate achieves a cell concentration of greater than 180 grams wet mass of cells per liter.

22. The process of claim 17, wherein the inoculum comprises about 5% to 10% of the volume of the first batch of cells.

23. A process for production of an encapsulated cell product, the process comprising the steps of:
a. mixing an aqueous solution with a concentrated encapsulation medium to form an encapsulation medium, wherein the mixing is performed without the addition of heat sufficient to sterilize the encapsulation medium;
b. mixing cells with the encapsulation medium without heating to form a cell encapsulation mixture; and
c. polymerizing the cell encapsulation mixture to produce an encapsulated cell product.

24. The process of claim 23, wherein a sterilization agent is added to the aqueous solution.

25. The process of claim 24, wherein sodium chlorite is the sterilization agent.

26. The process of claim 23, further comprising the step of adding antibiotics to the encapsulation medium.

27. The process of claim 23, further comprising the step of adding nutrients to the encapsulation medium.

28. The process of claim 23, further comprising the step of adding vitamins to the encapsulation medium.

29. The process of claim 23, further comprising the step of irradiating the concentrated encapsulation medium.

30. The process of claim 23, further comprising the step of irradiating the encapsulation medium.

31. The process of claim 23, wherein the mixing cells step is performed with a reciprocating shaker.

32. The process of claim 23, wherein the mixing cells step is performed with a reciprocating disk.

33. The process of claim 23, wherein the concentrated encapsulation medium comprises sodium alginate.

34. The process of claim 33, wherein the sodium alginate is mixed with the aqueous solution to produce an encapsulation medium with a viscosity of about 1000 to 3500 centistokes.

35. The process of claim 33, wherein the cell encapsulation mixture has a viscosity of about 1000 to 3500 centistokes.

36. The process of claim 33, wherein the encapsulation medium has a sodium alginate concentration of 3.0% or less weight per volume.

37. The process of claim 33, wherein the encapsulation medium has a sodium alginate concentration of 2.5% or less weight per volume.

38. The process of claim 33, wherein the encapsulation medium has a sodium alginate concentration of about 2.0% weight per volume.

39. An alginate bead for encapsulation of a cell, the bead comprising:
a. polymerized alginate in a concentration of 3% or less weight per volume; and
b. an encapsulated cell.

40. The alginate bead of claim 39, wherein the alginate concentration is 2.5% or less weight per volume.

41. The alginate bead of claim 39, wherein the alginate concentration is about 2.0% weight per volume.
